(19) United States
(12) Patent Application Publication
(10) Pub. No.: US 2010/0190257 A1
(43) Pub. Date: Jul. 29, 2010

(54) Self-Assembly of a Cell-Microparticle Hybrid

(75) Inventors: Aliasger K. Salem, Iowa City, IA (US); Yogita Krishnamachari, Iowa City, IA (US)

Correspondence Address: Dr. Aliasger K Salem;Yogita Krishnamachari, The University of Iowa 115 S Grand Avenue, Dept. of Pharmaceutics, Iowa City, IA, 52242, US

(73) Assignee: THE UNIVERSITY OF IOWA RESEARCH FOUNDATION

(21) Appl. No.: 12/359,336

(22) Filed: Jan. 25, 2009

(51) Int. Cl. C12N 15/87; C12N 11/02; C12N 11/08; C12N 11/06

(52) U.S. Cl. 435/455; 435/177; 435/180; 435/181

(57) ABSTRACT

The present invention provides a fabrication method for the formation of a cell-microparticle hybrid. A biotin-avidin binding system also employs the use of a biodegradable polymer and any cell type that self-assemble to form a hybrid system.

### BACKGROUND OF THE INVENTION

I. Field of Invention

The present invention relates to the fields of vaccine delivery and tissue engineering. More particularly the invention relates to the self-assembly of a cell-microparticle hybrid.

II. Related Art

The self assembly of building blocks into more complex structures has attracted increasing attention for use in the fabrication of higher order devices and structures. [1,2] Most published reports pertaining to the assembly of building blocks focus on the self-assembly of synthetic structures [2-5] via physical, covalent, or biological interactions. [6,7] For example, rods and spheres have been assembled onto substrates and with each other using electrostatic interactions or interactions involving DNA strands. [8-10] Indeed, combining biological and synthetic materials has become increasingly important for tissue engineering, advanced drug delivery, and the development of intelligent biosynthetic devices. [11-15].

Another area that has been garnering increasing attention is the co-delivery of antigen and adjuvant to the same antigen to generate a significantly stronger antigen-specific and immunostimulatory response [16-18]. Recent studies highlight the need for innovative strategies to co-deliver antigen and adjuvant that can additionally protect the adjuvant from enzymatic degradation, improve targeting by enhanced cellular uptake and hence provide long term immunity.

### SUMMARY OF THE INVENTION

In accordance with the present invention, there is provided a method for self-assembly of cell-microparticle hybrid comprising (a) of biotinylated surface of a transfected or non transfected cell, (b) biotinylated biodegradable microparticles and (c) self-assembly of this biological-synthetic hybrid by the addition of avidin as a bridging molecule. The polymeric microparticles used are biodegradable. The di-block co-polymer may be poly(lactic acid)-poly(ethylene glycol) (PLA-PEG) or poly(lactide-co-glycolide)-PEG (PLGA-PEG).

The method may comprise of (a) biotinylating cell surface. The biotinylation of cell surface may comprise of treating cell surface with sodium periodate to generate a cell surface comprising of non-native aldehydes and reacting the cell comprising non-native aldehydes with biotin hydrazide.

The method may further comprise of a step of synthesis of a biodegradable di-block co-polymer comprising of PLA-PEG conjugated to biotin. poly(lactic acid)-poly(ethylene glycol)-biotin(PLA-PEG-biotin, copolymer has been synthesized by reacting N-hydroxysuccinimide(NHS)-biotin with the amine terminus of bifunctional α-amine-x-hydroxy-PEG. PEG-biotin is further conjugated to PLA by ring opening polymerization in the presence of stannous octanoate as a catalyst.

The method may further comprise of step (b) fabrication of microparticles comprising the polymer in [0009] by a double emulsion solvent evaporation technique.

The method may further comprise of the final step of self-assembly of (a) biotinylated cells) and (b) biotinylated biodegradable microparticles by the addition of (c) avidin as a bridging molecule to serve as a self assembly scaffold for tissue engineering applications.

The method may further comprise of a step prior to (c) wherein, in (b) the microparticles may be loaded with molecules like growth factors, proteins, signaling molecules etc. that may provide stimulus for cell differentiation, maturation and proliferation when used as a tissue engineering building block.

In another embodiment, there is provided a method (d) for the self-assembled cell-microparticle hybrid to serve as an innovative vehicle for the co-delivery of antigen and immunostimulatory adjuvant to the same antigen presenting cell (APC).

The method (d) may further comprise of a step prior to (a), comprising transfection of the cells to express the desired protein of interest.

The method may further comprise of loading an immunostimulatory adjuvant in the microparticles during step (b).

The method may further comprise of (e) biotinylating the transfected cells by sodium periodate treatment as explained in (a). The cells sued may be irradiated tumor cells expressing antigen of interest to the desired application.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1: HEK-293 cells are treated with sodium periodate to convert native sialic residues into non-native aldehyde groups. The aldehyde groups on the surface of the cells are reacted with biotin hydrazide to produce biotinylated cells.

FIG. 2: Schematic of preparation of spatially controlled biotinylated cells on biodegradable templates. (a) Biotin is covalently attached to a-hydroxy-amine PEG. (b) Lactide is graft polymerized onto hydroxyl terminus of biotin-PEG-OH.

FIG. 3: Schematic depiction of the self-assembly of microparticle-cell hybrids.

FIG. 3: b) mean percentage of human embryonic kidney 293 (HEK293) cells with PLA-PEG-biotin microparticles adhering to the cell surface, c) mean number of microparticles binding per HEK293 cell, d) fluorescence microscopy image of PLA-PEG-biotin microparticles surface engineered with rhodamine-labeled avidin (Olympus BX40 555/580 nm, scale bar=4.5 lm), e) light microscopy image of poly(lactic acid)-poly(ethylene glycol)-biotin (PLA-PEG-biotin) microparticles bound to avidinylated HEK293 cells (scale bar=25 lm), f) PLA-PEG-biotin microparticles incubated with control HEK293 cells without any biotinylation (scale bar=30 lm), g) HEK293 cells in suspension treated with NaIO4 and biotin hydrazide and self-assembled with PLA-PEG-biotin microparticles (scale bar=5 lm), h) HEK293 cells in suspension treated with biotin hydrazide alone and self-assembled with PLA-PEG-biotin microparticles, i) control HEK293 cells in suspension that have not been treated with biotin hydrazide do not self-assemble upon incubation with PLA-PEG-biotin microparticles, j) typically non-adherent EG7 tumor cells treated with NaIO4 and biotin hydrazide efficiently assemble with PLA-PEG-biotin microparticles (scale bar=10 lm), k) SEM image of self-assembled microparticle-cell hybrids (scale bar=1.5 lm), l) fluorescence microscopy overlay image of HEK293 cells transfected with green fluorescent protein and assembled with PLA-PEG-biotin microparticles loaded with rhodamine 123 (Olympus BX40 555/580 nm, 494/518 nm, scale bar=1.5 lm) for the purpose of application of claim (2).

FIG. 4: HEK 293 cells transfected to express Green Fluorescent Protein

### DETAILED DESCRIPTION OF INVENTION

### I. The Present Invention

The inventors now describe a biotin-avidin interaction system that can be utilized to develop a cell-microparticle hybrid. Avidin and biotin are broadly used in biological analysis techniques such as immunoassays because they make a highly specific and stable complex. Avidin (MW approximately 68 kDa) is a glycoprotein with 4 subunits that can each bind a biotin (also known as vitamin H; MW 244.3) The affinity constant of the avidin-biotin complex is about 10¹⁵ M⁻¹ [19].

### [A] Method of Biotinylating Cell Surface

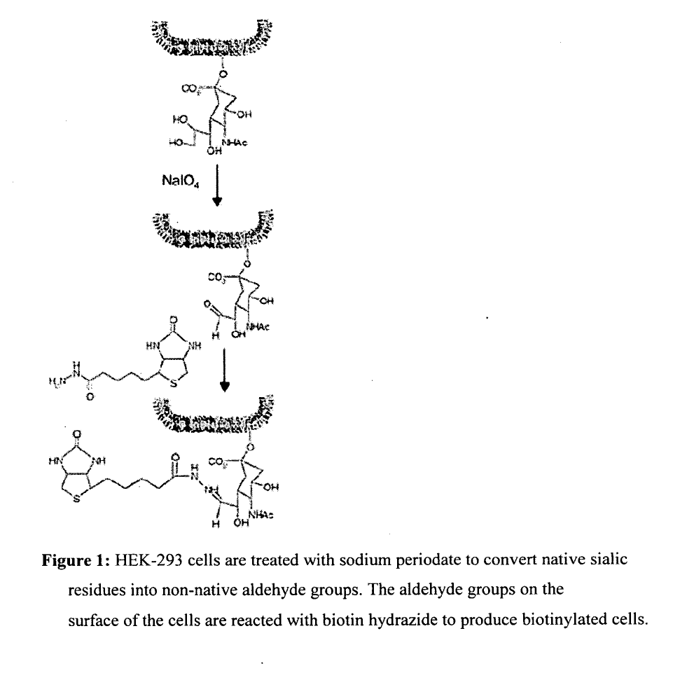

In this step of the process, human embryonic kidney 293 (HEK293, American Type Culture Collection (ATCC)) cells have been biotinylated. This has been achieved by converting native sialic acid residues on the cell surfaces into non-native aldehydes using a mild NaIO4 solution. Sialic acid is a ubiquitous terminal cell surface monosaccharide group with amplified expression in many cancers [20]. The aldehyde groups have been reacted with biotin-hydrazide to produce biotinylated cells. To achieve this objective, HEK293 cells have been grown to 60-65% confluence in 12-well plates. Cell culture media in the wells have been removed and replaced with freshly warmed media and further incubated for 1 h. Subsequently, the cells have been washed twice with phosphate buffered saline (PBS) and incubated with a 1 mM solution of NaIO4 in cold PBS for 20 min in the dark at 4° C. The HEK293 cells have been washed with PBS at pH 6.5 supplemented with 0.1% bovine calf serum (BCS) at room temperature. Next, the cells have been incubated with a 0.5 mM solution of biotin hydrazide (Sigma) in PBS (pH 6.5) for 90 min at room temperature. Then, the cells have been washed twice with PBS solution at pH 7.4 supplemented with 0.1% BCS. The process of cell surface biotinylation is depicted in FIG. 1.

### [B] Synthesis of Biotinylated-Biodegradable Polymer

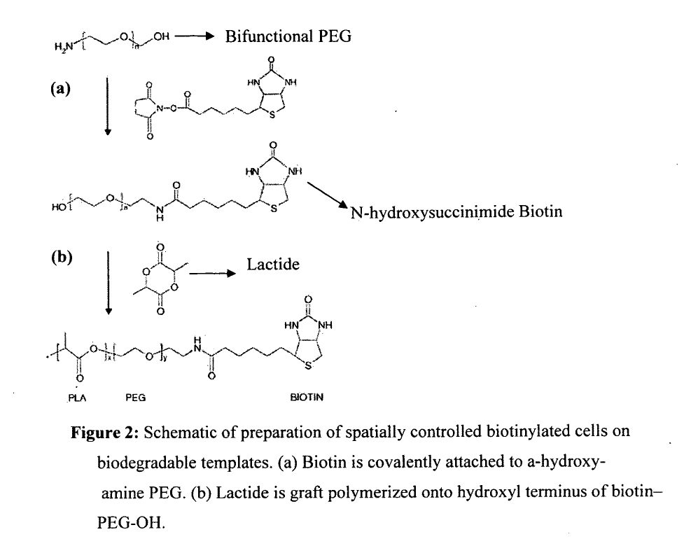

A biotinylated polylactic acid-polyethylene glycol copolymer is synthesized by reacting N-hydroxysuccinimide-biotin with the amine terminus of a bifunctional a-amino-hydroxy-polyethylene glycol that was prepared by reducing a-amine-o-carboxylic acid-polyethylene glycol in a 1M tetrahydrofuran-borane mixture (Sigma). Confirmation of the amide bond between the biotin and the PEG was observed by the appearance of a triplet at 7.8 ppm in 1H-NMR. Lactide was then graft polymerized onto the hydroxyl terminus of the a-biotin-o-hydroxy-polyethylene glycol in the presence of a stannous 2-ethyl hexanoate initiator as represented in FIG. 2. [21, 22]

### [C] Fabrication of PLA-PEG-Biotin Microparticles

PLA-PEG-biotin microparticles have been prepared using a double-emulsion solvent-evaporation approach [23]. This method utilizes three distinct phases, an inner water phase wherein the relevant proteins or drugs are entrapped, an intermediate organic phase composed of the polymer/methylene chloride solution, and an outer water phase containing an emulsifying agent. These microparticles can be loaded with a wide variety of drugs, proteins, or fluorescent molecules for imaging applications. [23] The particle size of these microparticles has been determined to be 1.4 μm from light scattering measurements using a Zetasizer Nano ZS instrument.

### [D] Self-Assembly of Cell-Microparticle Hybrid

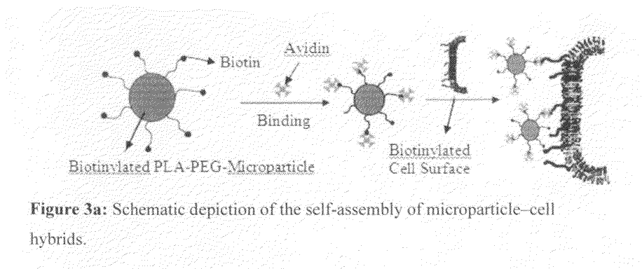

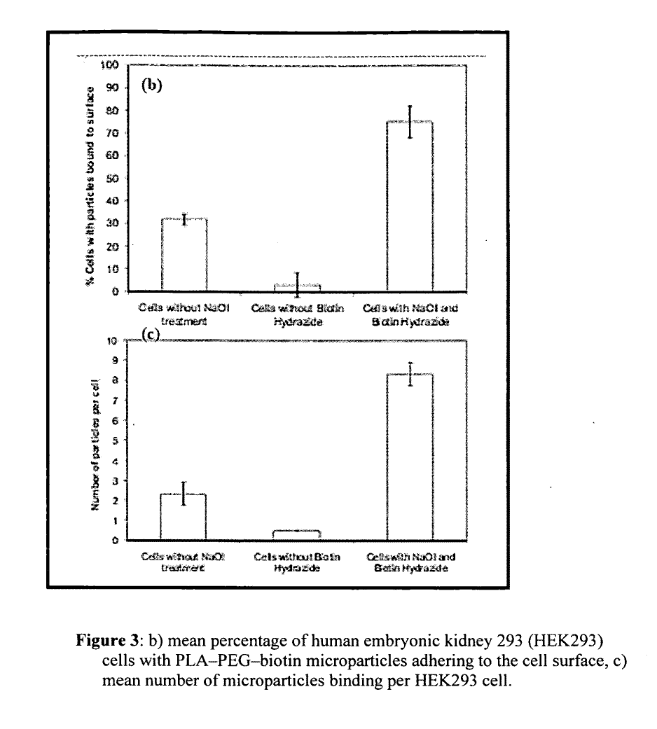

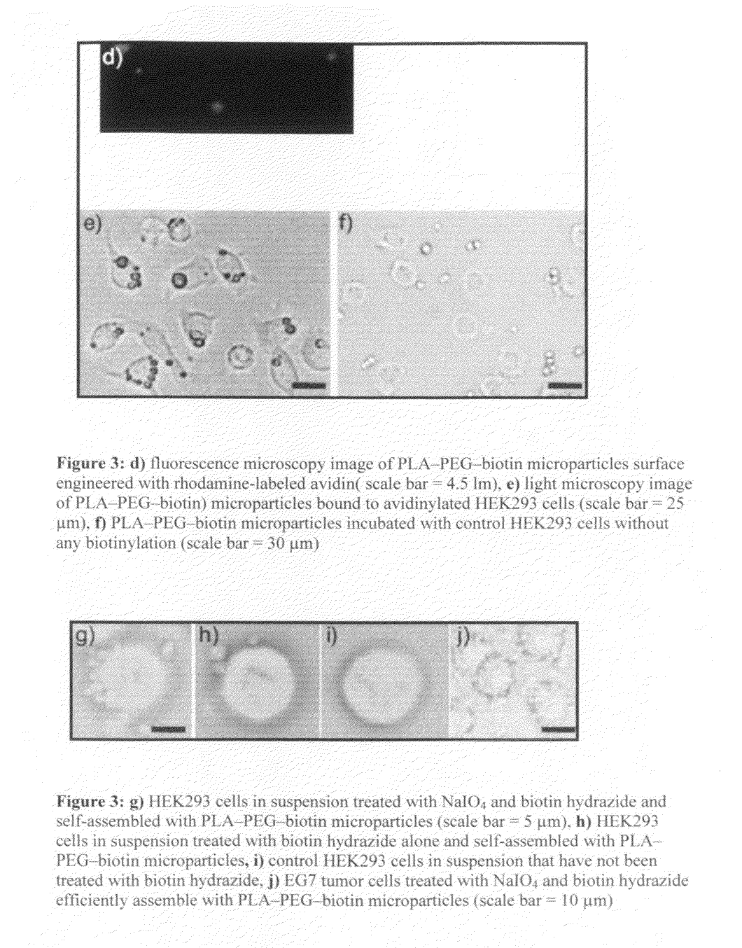

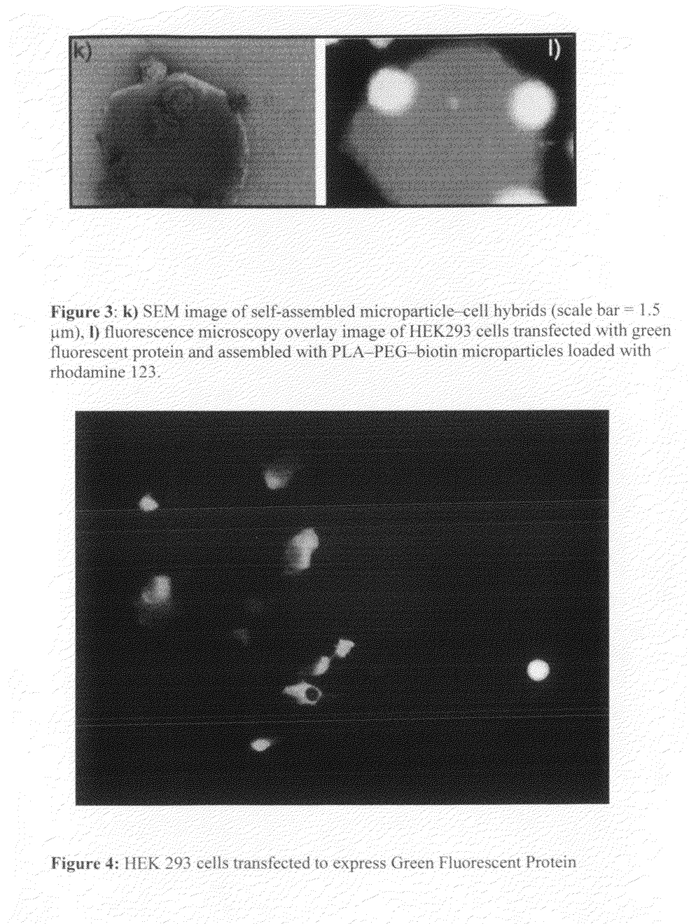

The biotinylated cells (step a) have been incubated with 1 mg mL⁻¹ of avidin-saturated biotinylated microparticles (step b) for 20 min at 4° C. Subsequently, the cells have been again washed twice with PBS (pH 7.4) prior to imaging by light microscopy (Olympus BX40). The degree of biotinylation on the surface of the HEK293 cells has been determined using the 2-(4-hydroxyazobenzene) benzoic acid (HABA)/avidin assay to be (3.6±0.45)×10⁹ biotin moieties per cell. The cell viability has been determined using trypan-blue measurements. Biotin-functionalized cells demonstrate a viability of 89.7% as compared to 97.51% for untreated cells. A self-assembled cell-microparticle hybrid is represented in FIGS. 3e, 3j, and 3k.

The inventors now describe another embodiment of biotin-avidin interaction system that can be utilized to develop a cell-microparticle hybrid as an innovative vaccine carrier for the co-delivery of antigen and immunostimulatory adjuvant to the same antigen presenting cell (APC).

### [E] Transfection of Cells Prior to Biotinylation to Express Desired Protein of Interest

In this step, 8×10⁴ HEK-293 cells were seeded on a 24-well plate 24 hours prior to transfection. Polyethyleneimine (PEI) was used as a non-viral polymeric carrier for the pDNA encoding green fluorescent protein (GFP). The transfection vehicle was incubated with the cells for 4 hours. At the end of 4 hours the cells were washed and fresh medium was added and cells were incubated at 37° C. for 44 hours. Success of transfection was assessed using an Olympus BX40 fluorescent microscope. Cells that were successfully transfected looked as represented in FIG. 4.

### [F] Fabrication of Rhodamine-Loaded PLA-PEG-Biotin Microparticles

The same procedure as described in (c) was followed with the additional step of addition of rhodamine 123 in dichloromethane along with the said polymer.

### [G] Self-Assembly of Transfected Cell-Rhodamine-Loaded Biodegradable Microparticle Hybrid

The same procedure as described in (d) was followed and the hybrid formed is represented in FIG. 4k.

### [H] Cell Types

Virtually any cell type and size can be attached to surfaces in accordance with the present invention. The cells may be prokaryotic or eukaryotic, plant, fungal, animal or human. The cells may be neuronal, endothelial or epithelial in origin, or may be lymphocytes, fibroblasts or myoblasts. Particular cells types include bacteria such as E. coli, Staphylococcus, myoblast precursors to skeletal muscle cells, neutrophils, erythroblast, osteoblasts, chondrocyte cartilage cells, basophil, eosinophils, adipocyte fat cells, invertebrate neurons (Helix aspera), mammalian neurons, adrenomedullary cells, melanocytes, or cancer cells. In addition, naturally occurring or genetically engineered cells, including those form plants, mammals, or invertebrates may be employed as well as mixtures of cells. A particularly useful source of cell lines may be found in ATCC Cell Lines and Hybridomas (8th ed., 1994), Bacteria and Bacteriophages (19th ed., 1996), Yeast (1995), Mycology and Botany (19th ed., 1996), and Protists: Algae and Protozoa (18th ed., 1993), each of which are herein incorporated by reference.

### Results

### EXAMPLE I

Biotin-functionalized cells demonstrate a viability of 89.7% as compared to 97.51% for untreated cells. FIG. 1d shows an image of PLA-PEG-biotin microparticles that have been incubated with HEK293 cells surface functionalized with avidin/biotin. The results of the control experiment in FIG. 1e shows that the microparticles do not bind as effectively to control HEK293 cells that have not been treated with biotin hydrazide. For control HEK293 cell samples that have not been treated with biotin hydrazide, it has been observed that the microparticles readily settle in areas where the non-confluent cells have not spread. The ability to specifically bind particles to non-confluent cells using receptor-mediated interactions has significant potential for improving in vitro drug and gene delivery. For example, biotinylated nanoparticles loaded or complexed with plasmid DNA could potentially significantly enhance the transfection efficiencies of avidin-biotin surface-engineered cells. To demonstrate that cell-microparticle hybrids can be prepared in solution, the cells have been trypsinized and treated with NaIO4 and biotin hydrazide as described above. Avidinylated PLA-PEG-biotin microparticles (1 mg mL⁻¹) have been self-assembled with the biotinylated cells (1×10⁵) by gently pipetting the two solutions into a single vial. Control experiments have also been performed wherein the cells are treated in exactly the same manner except for treatment with NaIO4. The results of the control experiments show a two- to three-fold reduction in the percentage of cells binding microparticles and a four-fold reduction in the number of particles bound per cell (FIGS. 3b and c). Control experiments where the cells have not been treated with biotin hydrazide indicate limited or no assembly of microparticle-cell hybrids (FIGS. 1e and h). This confirms that the self-assembly process arises from specific biotin-avidin receptor-mediated interactions (FIGS. 3b, c, e, j, k, and l).

### EXAMPLE II

To demonstrate the potential of our cell-microparticle hybrids in dual synthetic-biological drug and protein delivery applications, we have transfected non-adherent EL4 cells and adherent HEK293 cells with green fluorescent protein (GFP). Microparticles loaded with rhodamine 123 have been prepared using the double-emulsion solvent-evaporation technique and assembled with GFP-expressing cells using the biotin-avidin interaction. FIG. 31 shows a fluorescent overlay image of rhodamine-labeled microparticles efficiently assembled onto HEK293 cells expressing GFP. To demonstrate that this process is compatible with non-adherent cells transfected with antigenic proteins, EG7 cells (ATCC, 1×10⁵) have been engineered with biotin using the same procedure as described above for the HEK293 cells. The EG7 cells have been derived from the murine T-cell lymphoma EL4 cell line transfected with cDNA for a model protein antigen, ovalbumin. FIG. 3k shows that when the biotinylated EG7 cells are incubated with avidinylated PLA-PEG-biotin microparticles, cell-microparticle hybrids are readily constructed.

### Utility of Above Invention

Tissue Engineering Application

Cell transplants have been used clinically for bone marrow reconstitution for over 30 years. Cell transplants are now being tested for neurological disorders such as Parkinson's [24-26] and Huntington's diseases [27, 28]. In addition mimicking the local environment at the site of cell transplant by the presence of signaling molecules, growth factors and proteins can have specific mechanical and biological properties similar to the native extracellular matrix (ECM). The interactions between cells and ECM control cellular activities such as migration, proliferation, differentiation, gene expression, and organogenesis [29, 30]. Hence a hybrid system like the one developed by us can mimic the ECM of the host to the greatest extent and thus serve as an intelligent bio-synthetic building block in tissue engineering.

Innovative Vaccine Carrier

While CpG ODN an adjuvant, exhibits potent immunostimulatory effects, the rapid degradation and ineffective delivery into the intracellular compartments of APCs are major bottlenecks to improving its efficacy [31]. When antigen is administered alone, it elicits strong T_{H}2 type immune responses. A significant shift in the isotype of antibody response can be achieved by co-administering antigen and CpG ODN [32-35]. Addition of CpG ODN has been reported to result in a significant increase in production of IgG2a antibody, increasing the IgG2a: IgG1 ratio over nine-fold [12]. This enhanced T_{H}1 type immune response is essential for counteracting intracellular pathogens including choriomeningitis virus, Hepatitis B virus and tetanus toxoid. For example, co-administration of CpG ODN and hepatitis B surface antigen (HBsAg) vaccine to the same site of the muscle significantly enhanced the antibody response [36]. In contrast, when CpG ODN was administered separately following the administration of the vaccine, it did not induce any significant improvement in immunostimulatory effects over the administration of vaccine alone [36]. These studies highlight the importance of delivery devices that protect CpG ODN from enzymatic degradation, improve targeting of CpG ODN to the intracellular compartments of APCs, ensure that both CpG ODN and antigen are co-delivered to the same APC, and provide long term immunity. With this new hybrid system, cells can be transfected with granulocyte-macrophage colony-stimulating factor (GM-CSF) and self-assembled with microparticles loaded with immunostimulatory molecules such as CpG oligonucleotides as a new and potent vaccine for cancer delivering both antigen and adjuvant to the same APC.

### Materials and Methods

### [A] Synthesis of PLA-PEG-Biotin

a-hydroxy-x-amine PEG (1 g) was dissolved in a mixture of acetonitrile (2 mL, Aldrich), methylene chloride (1 mL, Aldrich), and Et3N (80 lL, Aldrich). After the addition of NHS-biotin (0.250 g, Sigma), the reactants were stirred overnight under argon. Subsequently, the reaction was worked-up by the slow addition of diethyl ether (40 mL, Aldrich) to precipitate the polymer. The polymer was reprecipitated from hot isopropyl alcohol (70° C., Aldrich). The reprecipitated polymer (350 mg) was then dried azeotropically and left under vacuum. Lactide (2 g, Purac Biochem bv) was added to biotin-PEG-OH (0.35 g) and diluted with 10 mL toluene and Sn(oct)2/toluene (0.1 g in 1 mL). The reaction mixture was then brought to reflux at 110° C. for 4 h under argon. The product was precipitated from a dichloromethane (DCM) solution into a cold stirring solution of diethyl ether and isolated by vacuum filtration. The final product was characterized by GPC and 1H NMR spectroscopy.

### [B] Preparation of PLA-PEG-Biotin Microparticles

PLA-PEG-biotin (50 mg) was dissolved in 5 mL of DCM. For rhodamine-loaded microparticles, 1 mg rhodamine 123 (Sigma) was also dissolved in 5 mL DCM. The polymer solution was then added to 500 lL of a 1% (w/v) PVA (Mw: 250 000, 88% hydrolyzed, Sigma) solution and ultrasonicated for 30 s. The primary emulsion was then added to 50 mL of 1% (w/v) PVA solution and homogenized further for 3 min at 13 500 rpm. The emulsion was then left stirring overnight over a magnetic stirrer to allow DCM to evaporate and to enable the formation of microparticles. The average diameter of the microparticles was 1.4 lm, as determined by measurements made using a Zetasizer NanoZS (Malvern Instruments) instrument.

### [C] Cell Culture

HEK293 cells and EG7/EL4 (CRL-2113/TIB-39) cells were obtained from ATCC (Manassas, Va.). The cells were cultured in Dulbecco's Modified Eagle's Medium (DMEM) obtained from Gibco BRL (Grand Island, N.Y.) supplemented with 10% fetal bovine serum (FBS), streptomycin at 100 lg mL-1, penicillin at 100 U mL-1, and 4 mM L-glutamine at 37° C. in a humidified 5% CO2-containing atmosphere. The HEK293 cells were passaged at pre-confluence every 4 days in a 1:4 ratio using 0.25% trypsin. Fresh DMEM medium was replenished every 2 days during cell culture. The EG7/EL4 cells were passaged every alternate day in a 1:3 ratio by aspirating two-thirds of the medium and replacing with fresh medium.

### [D] Amplification and Purification of Plasmid DNA

GFP-plasmids (Clontech) were transformed to Escherichia coli DH5a and amplified in Terrific Broth media at 37° C. overnight at a shaking speed of 300 rpm. The plasmid was purified using an endotoxin-free Qiagen Giga plasmid purification kit (Qiagen, Valencia, Calif.) according to the protocol provided by the manufacturer. Purified pDNA was dissolved in saline, and its purity and concentration were determined by UV absorbance at 260 and 280 nm.

### [E] Transfection of HEK293 and EL4 Cells:

HEK293 and EL4 cells were seeded into 24-well plates at a density of 8×104 cells per well 24 h before starting transfection. Each well of the 24-well plate was transfected with 0.5 mL reduced-serum Opti-MEM media (Gibco). Polyethyleneimine(PEI, 25 000 branched, Sigma)/pDNA complexes in a ratio of 5:1 comprising 5 lg PEI in 40 lL Opti-MEM and 1 lg DNA in 40 lL Opti-MEM were added to each well. After 4 h, the transfection media was removed and the cells were washed. After 2 days of further incubation in serum-containing media, the wells were washed with PBS and imaged live. The cells were then ready for use in microparticle-cell assembly experiments.

### [F] HABA/Avidin Assay

This analysis was carried out using a spectrophotometer (Spectramax 384 plus, Molecular Devices, CA) at a fixed wavelength of 500 nm. 180 lL of HABA/avidin reagent was added to a 96-well plate and three readings of absorbance were recorded with an average read time of 0.5 s. After the initial recording, 120 lL of the supernatant collected following cell treatment with biotin hydrazide was added to each of the 96-well plates. Three sets of absorbance readings were recorded again after allowing 3 min for reaction. The absorbance decreased proportionately depending on the amount of biotin present on the cell surface because the biotin displaces HABA owing to its higher affinity for avidin. The changes in absorbance were used in conjunction with a calibration curve to calculate the extent of biotinylation of the cell surface.

### [G] Scanning Electron Microscopy (SEM)

Cell-microparticle hybrids were seeded onto a poly(L-lysine) coated (1 lg cm-2) coverslip and fixed with 2.5% glutaraldehyde solution. After 1 h, the hybrids were washed twice with a 0.2% sodium cacodylate buffer solution and dehydrated with 25, 50, 75, 95% (4 min each), and 100% ethanol (10 min each) solutions. The cell-microparticle hybrids were then treated with hexadimethylsilazane (HDMS) for 10 min and dried for 3 h. The samples were then sputter coated and visualized using SEM (Hitachi S4800).

### REFERENCES

[1] A. K. Boal, F. Ilhan, J. E. DeRouchey, T. Thurn-Albrecht, T. P. Russell, V. M. Rotello, Nature 2000, 404, 746.
[2] N. I. Kovtyukhova, T. E. Mallouk, Chem. Eur. J. 2002, 8, 4355.
[3] A. K. Boal, M. Gray, F. Ilhan, G. M. Clavier, L. Kapitzky, V. M. Rotello, Tetrahedron 2002, 58, 765.
[4] B. L. Frankamp, A. K. Boal, V. M. Rotello, J. Am. Chem. Soc. 2002, 124, 15146.
[5] A. K. Boal, B. L. Frankamp, O. Uzun, M. T. Tuominen, V. M. Rotello, Chem. Mater. 2004, 16, 3252.
[6] C. J. Orendorff, P. L. Hankins, C. J. Murphy, Langmuir 2005, 21, 2022.
[7] R. J. Thibault, 0. Uzun, R. Hong, V. M. Rotello, Adv. Mater. 2006, 18, 2179.
[8] T. H. Galow, A. K. Boal, V. M. Rotello, Adv. Mater. 2000, 12, 576.
[9] S. Srivastava, B. Samanta, P. Arumugam, G. Han, V. M. Rotello, J. Mater. Chem. 2007, 17, 52.
[10] J. K. N. Mbindyo, B. D. Reiss, B. R. Martin, C. D. Keating, M. J. Natan T. E. Mallouk, Adv. Mater. 2001, 13, 249.
[11] S. Raghavan, C. S. Chen, Adv. Mater. 2004, 16, 1303.
[12] W. Sun, J. E. Puzas, T. J. Sheu, X. Liu, P. M. Fauchet, Adv. Mater. 2007, 19, 921.
[13] R. Bhattacharya, C. R. Patra, R. Verma, S. Kumar, P. R. Greipp, P. Mukherjee, Adv. Mater. 2007, 19, 711.
[14] K. Y. Lee, H. J. Kong, R. G. Larson, D. J. Mooney, Adv. Mater. 2003, 15, 1828.
[15] M. P. Lutolf, G. P. Raeber, A. H. Zisch, N. Tirelli, J. A. Hubbell, Adv. Mater. 2003, 15, 888.
[16]A. K. Salem, C. F. Hung, T. W. Kim, T. C. Wu, P. C. Searson, K. W. Nanotechnology 16(4) (2005) 484-487.
[17]A. D. Sandier, H. Chihara, G. Kobayashi, X. Y. Zhu, M. A. Miller, D. L. Scott, A. M. Krieg, Cancer Research 63(2) (2003) 394-399.
[18]G. J. Weiner, H. M. Liu, J. E. Wooldridge, C. E. Dahle, A. M. Krieg, Proceedings of the National Academy of Sciences of the United States of America 94(20) (1997) 10833-10837.
[19] Green N. Mex. Avidin. Adv Protein Chem 1975;29:85-133
[20] J. A. Prescher, D. H. Dube, C. R. Bertozzi, Nature 2004, 430, 873.
[21] J. Sinclair, A. K. Salem, Biomaterials 2006, 27, 2090.
[22] A. K. Salem, S. M. Cannizzaro, M. C. Davies, S. J. B. Tendler, C. J. Roberts, P. M. Williams, K. M. Shakesheff, Biomacromolecules 2001, 2, 575.
[23] X. Q. Zhang, C. E. Dahle, N. K. Baman, N. Rich, G. J. Weiner, A. K. Salem, J. Immunotherapy 2007, 30, 469.
[24]. Freed, C. R. et al. N. Engl. J. Med. 8, 710-719 (2001).
[25]. Freed, C. R. et al. N. Engl. J. of Med. 327, 1549 (1992).
[26]. Spencer, D. D. et al. N. Engl. J. Med. 327, 1541-1548 (1992).
[27]. Kordower, J. H. et al. N. Engl. J. Med. 332, 1118 (1995).
[28]. Philpott, L. M. et al. Cell Transplant. 6, 203-212 (1997).
[29] Hubbell J A Bio-Technology June 1995; 13(6):565-576
[30] Langer R, Vacanti J P. Science May 1993; 260(5110):920-926
[31] H. Sands, L. J. Goreyferet, A. J. Cocuzza, F. W. Hobbs, D. Chidester, G. L. Trainor, Molecular Pharmacology 45(5) (1994) 932-943.
[32] R. S. Chu, O. S. Targoni, A. M. Krieg, P. V. Lehmann, C. V. Harding, Faseb Journal 12(4) (1998) A612-A612.
[33] M. J. McCluskie, H. L. Davis, Cutting edge: Journal of Immunology 161(9) (1998) 4463-4466.
[34] A. Oxenius, M. M. A. Martinic, H. Hengartner, P. Klenernan, Journal of Virology 73(5) (1999) 4120-4126.
[36] G. K. Mutwiri, A. K. Nichani, S. Babiuk, L. A. Babiuk, Journal of Controlled Release 97(1) (2004) 1-17.

1) A method for the preparation of a synthetic biodegradable microparticle-cell hybrid for the purpose of tissue engineering purposes.

2) A method stated in claim (1) modified for the preparation of a synthetic biodegradable microparticle-transfected cell hybrid to serve as an innovative carrier for vaccines for co-delivering antigen and immunostimulatory adjuvant.

3) The method of claim (1), wherein the first step comprises of biotinylating cell surface.

4) The method of claim (3), wherein biotinylating the said cell surface comprises treating the cell surface with sodium periodate to generate a cell comprising non-native aldehydes following the oxidation of cell surface sialic acid residues and reacting said cell surface comprising non-native aldehydes with biotin hydrazide.

5) The method of claim (3), wherein, alternatively biotinylating said cell surface comprises treating cell surface with sulfo-N-hydroxy succinimide biotin, to biotinylate the cell surface by N-hydroxy succinimide conjugation to lysine residues on cell surface.

6) The method of claim (3), wherein the hybrid employs the use of a biodegradable polymer.

7) The method of claim (6), wherein the said biodegradable polymer may be di-block poly (lactic acid)-poly (ethylene glycol);PLA-PEG or poly (lactide co-glycolide)-poly(ethylene glycol) polymer conjugated to biotin.

8) The method of claim (7), wherein the said biodegradable PLA_PEG-Biotin co-polymer has been synthesized by reacting N-hydroxy succinimide biotin with the amine terminus of bi-functional PEG and then conjugating it to PLA by ring opening polymerization in the presence of stannous octanoate as a catalyst.

9) The method of claim (8), wherein the synthesized biodegradable polymer is fabricated as microparticles.

10) The method of claim (9), wherein the microparticles are fabricated by double emulsion-solvent evaporation technique.

11) The method of claims (1) wherein, the biotinylated microparticles in claim (9) have been self-assembled with biotinylated cells in claim (4) using avidin as a bridging molecule.

12) The method in claim (2), wherein the microparticles can be loaded with immunostimulatory molecules.

13) The method in claim (2), wherein the biotinylated microparticles in claim (9) have been loaded with rhodamine as a model loading molecule.

14) The method in claims (1) and (2) wherein the cell populations are naturally adherent or non-adherent.

15) The method in claim (14) wherein the cell population comprises either an endothelial, epithelial or a lymphocyte cell population.

16) The method in claim (2) wherein, the cells are transfected prior to biotinylation to express the desired protein of interest.

17) The method in claim (16), wherein the cells can be transfected to express GMC_SF to serve as an antigenic carrier for the purpose of claim (2).

18) The method of claim (17), wherein, the cells were transfected to express green fluorescent protein (QFP) for demonstration purposes.
